(19) United States
(12) Patent Application Publication
(10) Pub. No.: US 2023/0120061 A1
(43) Pub. Date: Apr. 20, 2023

(54) METHODS FOR ALTERING TIME TO FLOWERING IN RUBUS PLANTS

(71) Applicant: Pairwise Plants Services, Inc., Durham, NC (US)

(72) Inventor: Joel Reiner, Raleigh, NC (US)

(21) Appl. No.: 17/874,361

(22) Filed: Jul. 27, 2022

Related U.S. Application Data
(60) Provisional application No. 63/226,356, filed on Jul. 28, 2021.

(51) Int. Cl. A01H 1/00; A01H 6/74; A01H 5/08

(52) U.S. Cl. CPC A01H 1/1215; A01H 1/106; A01H 6/7499; A01H 5/08

(57) ABSTRACT

This invention relates to methods for modifying flowering time and timing of fruit production in a Rubus plant. In addition, the invention relates to plants produced by the methods described herein.

### STATEMENT OF PRIORITY

This application claims the benefit, under 35 U.S.C. § 119 (e), of U.S. Provisional Application No. 63/226,356, filed on Jul. 28, 2021, the entire contents of which is incorporated by reference herein.

### FIELD OF THE INVENTION

This invention relates to methods for modifying flowering time and timing of fruit production in a Rubus plant. In addition, the invention relates to plants produced by the methods described herein.

### BACKGROUND OF THE INVENTION

Rubus plants that flower on a floricane, known as floricane fruiting varieties or cultivars, require a specific set of conditions in order to flower and produce fruit. These conditions are met by producing and maturing a primocane in year 1 followed by an inductive period, dormancy and a floricane with flowers and fruit the subsequent growing season. These specific requirements make evaluation of flowering and fruit both a time-consuming process requiring at least two years of growth and a complex process in which specific environmental requirements need to be met.

In addition to being a long and resource intensive process, the flowering and fruiting of Rubus is also a process that is difficult to adapt to a continual production pipeline under natural (outdoor) conditions. A typical growing season has a determined and optimal planting, maturing, inductive, dormant, and flowering season. For the plants that miss the narrow planting window, additional time must be added (in some cases, up to a year) to the evaluation timeline. The long feedback loop from plant to obtaining flowering and fruiting data adds significant time to the process of developing new varieties and cultivars.

The present invention overcomes the shortcomings in the art by providing methods that reduce the time to flowering in a Rubus plant.

### Summary of Example Embodiments

One aspect of the invention provides a method of reducing time to flowering of a Rubus plant, the method comprising: (a) growing a Rubus plant under conditions comprising at least 4 hours of continuous darkness in a 24-hour period and a temperature of at least 21° C. to produce a Rubus plant having more than 14 nodes; (b) growing the Rubus plant having more than 14 nodes of (a) under induction conditions comprising a temperature of at least 9° C. and a day length of at least 8 continuous hours in a 24-hour period to produce an induced Rubus plant; (c) placing the induced Rubus plant of (b) under dormancy conditions comprising a 24-hour dark period (i.e., continuous darkness) and a temperature of about 0° C. to about 7° C. to produce a dormant Rubus plant; (d) optionally, growing the dormant Rubus plant of (c) under conditions comprising at least 4 hours of continuous darkness in a 24-hour period and a temperature of at least 21° C. to produce a Rubus plant that is flowering, thereby reducing the time to flowering of a Rubus plant.

Another aspect of the invention provides a method of reducing time to flowering of a primocane flowering Rubus variety or cultivar, comprising: (a) growing the primocane flowering Rubus variety or cultivar under conditions comprising at least 4 hours of continuous darkness in a 24-hour period and a temperature of at least 21° C. to produce a primocane flowering Rubus variety or cultivar having more than 14 nodes; (b) growing the primocane flowering Rubus variety or cultivar having more than 14 nodes of (a) under induction conditions comprising a temperature of at least 9° C. and a day length of at least 8 continuous hours in a 24-hour period to produce an induced primocane flowering Rubus variety or cultivar; and (c) growing the induced primocane flowering Rubus variety or cultivar of (b) under conditions comprising at least 4 hours of continuous darkness in a 24-hour period and a temperature of at least 21° to produce a primocane flowering Rubus variety or cultivar having flowers, thereby reducing the time to flowering of a primocane flowering Rubus variety or cultivar.

An additional aspect of the invention provides a method of producing a Rubus plant having reduced time to flowering, the method comprising: (a) growing a Rubus plant under conditions comprising at least 4 hours of continuous darkness in a 24-hour period and a temperature of at least 21° C. to produce a Rubus plant having more than 14 nodes; (b) growing the Rubus plant having more than 14 nodes of (a) under induction conditions comprising a temperature of at least 9° C. and a day length of at least 8 continuous hours in a 24-hour period to produce an induced Rubus plant; (c) placing the induced Rubus plant of (b) under dormancy conditions comprising a 24-hour dark period (i.e., continuous darkness) and a temperature of about 0° C. to about 7° C. to produce a dormant Rubus plant; (d) optionally, growing the dormant Rubus plant of (c) under conditions comprising at least 4 hours of continuous darkness in a 24-hour period and a temperature of at least 21° C. to produce a Rubus plant that is flowering, thereby producing a Rubus plant having reduced time to flowering

Further provided is a method of producing a primocane flowering Rubus plant having reduced time to flowering, the method comprising: (a) growing the primocane flowering Rubus variety or cultivar under conditions comprising at least 4 hours of continuous darkness in a 24-hour period and a temperature of at least 21° C. to produce a primocane flowering Rubus variety or cultivar having more than 14 nodes; (b) growing the primocane flowering Rubus variety or cultivar having more than 14 nodes of (a) under induction conditions comprising a temperature of at least 9° C. and a day length of at least 8 continuous hours in a 24-hour period to produce an induced primocane flowering Rubus variety or cultivar; and (c) growing the induced primocane flowering Rubus variety or cultivar of (b) under conditions comprising at least 4 hours of continuous darkness in a 24-hour period and a temperature of at least 21° to produce a primocane flowering Rubus variety or cultivar having flowers, thereby producing a primocane flowering Rubus variety or cultivar having reduced time to flowering.

In addition, the present invention provides Rubus plants produced by the methods described herein.

These and other aspects of the invention are set forth in more detail in the description of the invention below.

### BRIEF DESCRIPTION OF THE DRAWINGS

FIG. 1 is a drawing of a Rubus plant showing the various structures associated with flowering and fruiting including primocanes, floricanes, and fruiting laterals.

FIG. 2 is a drawing of a plant stem showing the nodes, buds, and internodes on a plant stem.

FIG. 3 shows the measurement of the number of nodes produced over time on a selected stem of a Rubus Experimental/Precommercial Variety #1. CE43380, CE43375, CE43377, CE43381, CE43382, and CE433791 are individual plants of this variety.

FIG. 4 shows the measurement of the number of nodes produced over time on a selected stem of a Rubus Experimental/Precommercial Variety #2. CE43446, CE43442, CE43445, CE43443, CE43444, and CE43441 are individual plants of this variety.

FIG. 5 shows the measurement of the number of nodes produced over time on a selected stem of a Rubus Experimental/Precommercial Variety #3. CE43469, CE43466, and CE43459 are individual plants of this variety.

FIG. 6 shows the measurement of the number of nodes produced over time on a selected stem of a Rubus d Commercial Variety #1. CE46603 and CE46600 are individual plants of this variety.

FIG. 7 shows the measurement of the number of nodes produced over time on a selected stem of a Rubus Commercial Variety #2. CE46606, CE46605, CE46607, CE46609, and CE46608 are individual plants of this variety.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range of 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In some embodiments, the reduction can result in no or essentially no (i.e., an insignificant amount, e.g., less than about 10% or even 5%) detectable activity or amount.

“Natural,” “native” or “wild type” conditions for flowering in plants, e.g., Rubus plants, refer to those conditions that are necessary for a plant to flower and produce fruit when cultivated in a field, or grown in a growth environment which does not substantially differ from field grown conditions, or grown wild without manipulation by humans.

As used herein, the terms “cultivar” and “variety” refer to a group of similar plants that by structural or genetic features and/or performance can be distinguished from other varieties or cultivars within a species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including, but not limited to, the cross between two inbred lines. In some embodiments, a Rubus hybrid can include but is not limited to, a hybrid between any of the Rubus species of Rubus allegheniensis (Allegheny blackberry), Rubus armeniacus (Himalayan blackberry), Rubus canadensis (Canadian blackberry), Rubus fruticosus agg. (European blackberry), Rubus laciniatus (cutleaf evergreen blackberry), Rubus pensilvanicus (Pennsylvania blackberry), Rubus trifidus (Japanese blackberry), or Rubus ursinus (trailing blackberry).

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

As used herein a “control plant” (e.g., control Rubus plant) means a plant (e.g., a Rubus plant) that is the same species, variety or cultivar as the Rubus plant that is contacted or treated with or exposed to the methods of the invention, but which has not been exposed to the methods (e.g., the conditions) of the invention. For example, a control for a floricane flowering variety of blackberry is that same blackberry variety that has not been contacted with the methods of the invention (e.g., exposed to the conditions and methods of the invention as described herein).

As used herein, “contact,” “contacting,” “contacted,” and grammatical variations thereof, refer to placing the components of a desired reaction together under conditions suitable for carrying out the desired reaction. As an example, a Rubus plant may be contacted with the light and temperature conditions as described herein to alter the timing of flowering and fruiting of the Rubus plant.

As used herein, the term “continuous” means without stopping (uninterrupted) for the designated period of time.

The term “plant part,” as used herein, includes but is not limited to reproductive tissues (e.g., petals, sepals, stamens, pistils, receptacles, anthers, pollen, flowers, fruits, flower bud, ovules, seeds, embryos); vegetative tissues (e.g., petioles, stems (e.g., primocane, floricane), roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, collenchyma cells, sclerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell. A “protoplast” is an isolated plant cell without a cell wall or with only parts of the cell wall. Thus, in some embodiments of the invention, a cell of a Rubus plant or plant part includes, but is not limited to, a root cell, a leaf cell, a tissue culture cell, a seed cell, a flower cell, a fruit cell, a pollen cell, and the like. In some aspects of the invention, the plant part can be a plant germplasm.

“Plant cell culture” means cultures of plant units such as, for example, protoplasts, cell culture cells, cells in plant tissues, pollen, pollen tubes, ovules, embryo sacs, zygotes, and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A “vegetative node” or a “node” as used herein is an area on a stem where buds are located (see, e.g., FIG. 2). It is a site of great cellular activity and growth, where small buds develop into leaves, stems, or flowers.

A “bud”, as used herein, is an undeveloped shoot from which leaves or flower parts grow. The buds of temperate-zone trees and shrubs typically develop a protective outer layer of small, leathery scales. Annual plants and herbaceous perennials have naked buds with green, somewhat succulent, outer leaves.

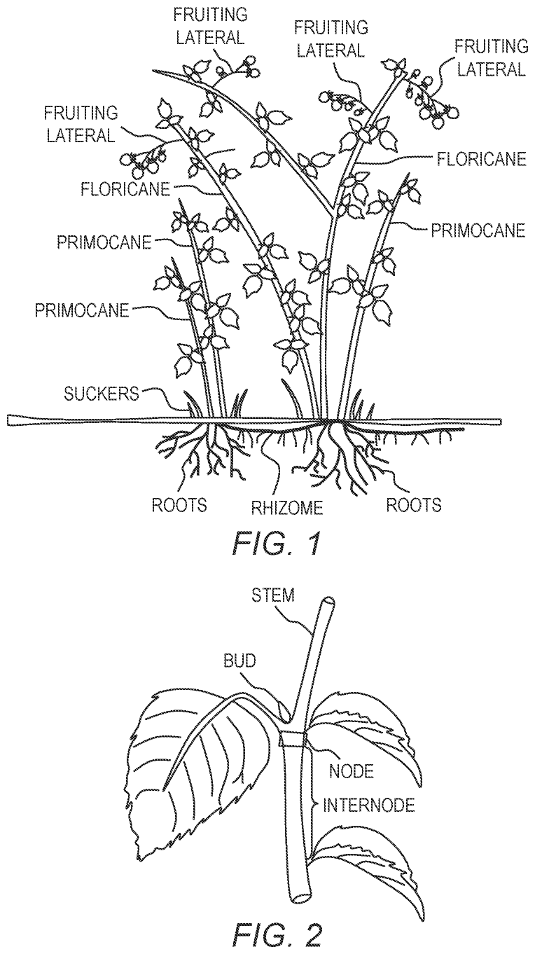

As is known in the art, depending on the variety or cultivar, a Rubus plant may flower on a floricane or a primocane stem. Those Rubus plants that are “floricane flowering” or “floricane fruiting” naturally flower and fruit on old wood, e.g., wood produced in the growing season prior to flowering, see, FIG. 1. In contrast, Rubus plants that are “primocane flowering” or “primocane fruiting” naturally flower and fruit on new wood, e.g., wood produced in the same growing season as flowering occurs. In particular for floricane flowering plants, a significant amount of time must be added to a process for evaluating new cultivars and varieties due to the long time period needed for flowering and fruiting. To address this issue, the present invention provides methods for reducing the time to flowering and fruiting in Rubus spp. plants for both floricane and primocane flowering plants, thereby reducing the time necessary for evaluating new Rubus cultivars and varieties.

Accordingly, a method of reducing time to flowering of a Rubus plant is provided, the method comprising: (a) growing a Rubus plant under conditions comprising at least 4 hours of continuous darkness in a 24-hour period and a temperature of at least 21° C. to produce a Rubus plant having more than 14 nodes; (b) growing the Rubus plant having more than 14 nodes of (a) under induction conditions comprising a temperature of at least 9° C. and a day length of at least 8 continuous hours in a 24-hour period to produce an induced Rubus plant; (c) placing the induced Rubus plant of (b) under dormancy conditions comprising a 24-hour dark period (i.e., continuous darkness) and a temperature of about 0° C. to about 7° C. (e.g., about 0, 1, 2, 3, 4, 5, 6, or 7° C., or any range or value therein) to produce a dormant Rubus plant; and/or (d) optionally, growing the dormant Rubus plant of (c) under conditions comprising at least 4 hours of continuous darkness in a 24-hour period and a temperature of at least 21° C. to produce a Rubus plant that is flowering, thereby reducing the time to flowering of a Rubus plant. In some embodiments, a method of producing a Rubus plant having reduced time to flowering is provided, the method comprising: (a) growing a Rubus plant under conditions comprising at least 4 hours of continuous darkness in a 24-hour period and a temperature of at least 21° C. to produce a Rubus plant having more than 14 nodes; (b) growing the Rubus plant having more than 14 nodes of (a) under induction conditions comprising a temperature of at least 9° C. and a day length of at least 8 continuous hours in a 24-hour period to produce an induced Rubus plant; (c) placing the induced Rubus plant of (b) under dormancy conditions comprising a 24-hour dark period (i.e., continuous darkness) and a temperature of about 0° C. to about 7° C. to produce a dormant Rubus plant; and/or (d) optionally, growing the dormant Rubus plant of (c) under conditions comprising at least 4 hours of continuous darkness in a 24-hour period and a temperature of at least 21° C. to produce a Rubus plant that is flowering, thereby producing a Rubus plant having reduced time to flowering.

In some embodiments, the “more than 14 nodes” on the stem(s) of the Rubus plant produced in (b) is about 15 to about 30 nodes (e.g., about 15, 16, 17, 18, 19, 20, 21, 22, 23, 24, 25, 26, 27, 28, 29, or 30 nodes, or any range or value therein). In some embodiments, the dormancy conditions of (c) comprise a temperature of about 3° C. to about 5° C. (e.g., about 3° C., 4° C., or 5° C., or any range or value therein), optionally the dormancy conditions of (c) comprise a temperature of about 4° C. In some embodiments, the at least 4 hours of continuous darkness in (a) and/or (d) may be about 4 hours to about 6 hours of continuous darkness (e.g., about 4, 4.25, 4.5, 4.75, 5, 5.25, 5.5, 5.75, or 6 hours of continuous darkness, or any range or value therein). In some embodiments, the temperature of at least 21° C. in (a) and/or (d) may be a temperature of about 21° C. to about 30° C. (e.g., about 21, 22, 23, 24, 25, 26, 27, 28, 29, 30° C., or any range or value therein).

In some embodiments, the temperature of at least 9° C. in the induction conditions of (b) may be about 9° C. to about 14° C. (e.g., about 9, 10, 11, 12, 13, or 14° C., or any range or value therein). In some embodiments, the day length of at least 8 continuous hours in a 24-hour period for in the induction conditions of (b) may be about 8 continuous hours to about 12 continuous hours (e.g., about 8, 8.25, 8.5, 8.75, 9, 9.25, 9.5, 9.75, 10, 10.25, 10.5, 10.75, 11, 11.25, 11.5, 11.75, or 12 hours, or any range or value therein) in a 24-hour period. In some embodiments, the day length of at least 8 continuous hours in a 24-hour period of (b) (e.g., about 8 hours to about 12 hours) includes light that is supplemented with far-red light (e.g., a wavelength of about 700 nm to about 800 nm).

In some embodiments, the Rubus plant is grown under the conditions of (a) for about 25 days to about 200 days (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, or 200 days, or any range or value therein, e.g., about 25, 26, 27, 28, 29, 30, 31, 32, 33, 34, 35, 36, 37, 38, 39, 40, 41, 42, 43, 44, 45, 46, 47, 48, 49, 50 days to about 100, 105, 110, 115, 120, 125, 130, 135, 140, 145, 150, 155, 160, 165, 170, 175, or 180 days or any range or value therein, optionally, for about 30, 31, 32, 33, 34, 35, 36, 37, 38, or 39 days to about 40, 41, 42, 43, 44, 45, 46, 47, 48, 49, 50, 51, 52, 53, 54, 55, 56, 57, 58, 59, or 60 days or any range or value therein. In some embodiments, Rubus plants may begin to be exposed to the conditions of (a) at about the time that the first set of true leaves emerge.

In some embodiments, the Rubus plant having more than 14 nodes is grown under the induction conditions of (b) for about 30 consecutive days to about 60 consecutive days (e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, e.g., about 1 to 2 months) and/or is grown under the dormancy conditions of (c) for about 1 day to about 6 months (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 81, 85, 90, 95, 100, 105, 110, 115 days to about 120, 125, 130, 135, 140, 145, 150, 155, 160, 165, 170, 175, 180, or 185 days, or any range or value therein, about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 81, 85, 90, 95, 100, 105, 110, 115, 120, 125, 130, 135, 140, 145, 150, 155, 160, or 165 days, about 120, 125, 130, 135, 140, 145, 150, 155, 160, 165, 170, 175, 180, or 185 days, or any range or value therein, or about 10 days to about 100 days, e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 81, 85, 90, 95, or 100 days).

In some embodiments, when the Rubus plant having more than 14 nodes is grown under the conditions of (d), the Rubus plant is grown under the conditions of (d) for about 1 day to about 6 months (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 81, 85, 90, 95, 100, 105, 110, 115 consecutive days to about 120, 125, 130, 135, 140, 145, 150, 155, 160, 165, 170, 175, 180, or 185 consecutive days, or any range or value therein, e.g., about 120, 125, 130, 135, 140, 145, 150, 155, 160, 165, 170, 175, 180, or 185 consecutive days, or any range or value therein, or e.g., about 10 consecutive days to about 100 consecutive days, e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 81, 85, 90, 95, or 100 consecutive days).

In some embodiments, a Rubus plant growing under the induction conditions of (b) may exhibit exponential growth for about 25 to about 60 days (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, e.g., about 3 weeks to about 2 months). In some embodiments, the Rubus plant growing under the induction conditions of (b) is moved to or placed under the dormancy conditions of (c) when exponential growth shifts to stationary growth (e.g., when the growth of the Rubus plant shifts to stationary growth). As used herein, “exponential growth” is a faster rate of growth than “stationary growth,” wherein the rate of growth is measured as an increase in stem length (internode length) over time and the increase in stem length (internode length) over time is greater for exponential growth than for stationary growth. Thus, in some embodiments, the Rubus plant is transferred to the dormancy conditions of (c) when the increase in stem length over time of the Rubus plant growing under the induction conditions of (b) slows and shifts to stationary growth. As used herein, “exponential growth,” and “stationary growth” for a Rubus plant is measured by selecting at least one stem on the plant and determining the number of nodes that develop on that at least one stem over time. In some embodiments, “exponential growth” of the Rubus stem is production of about one node per stem (e.g., vegetative node) (per stem measured) every one day to every three days (e.g., in about every 1 day, about every 2 days, about every 3 days, or any combination thereof, e.g., a node is produced about every 72 hours or less; e.g., about every 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 hours, or any range or value therein, e.g., about every 24, 25, 26, 27, 28, 29, 30, 31, 32, 33, 34, or 35 hours to about every 40, 41, 42, 41, 42, 43, 44, 45, 46, 47, 48, 49, 50, 51, 52, 53, 54, 55, 56, 57, 58, 59, 60, 61, 62, 63, 64, 65, or 66 hours, or any range or value therein, or combination thereof) and “stationary growth” of the Rubus stem is production of about one node (per stem measured) every three or more days (e.g. about every 3 days, about every 4 days, about every 5 days, about every 6 days or more, or any combination thereof, e.g., a node is produced about every 72 hours or more, e.g., about every 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 124, 125, 126, 127, 128, 129, 130, 131, 132, 133, 134, 135, 136, 137, 138, 139, 140, 141, 142, 143, or 144 hours, or any range or value therein, e.g., about every 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 124, 125, 126, 127, 128, 129, 130, 131, 132, 133, 134, 135, 136, 137, 138, 139, 140, 141, 142, 143, or 144 hours, or any range or value therein, or combination thereof). In some embodiments, a Rubus plant growing under the conditions of (b) may be shifted to the dormancy conditions of (c) at about 25 days to about 60 days after being exposed to the inductions conditions of (b) (e.g., at about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, about 25 days to about 9 weeks, e.g., at about 3 weeks to about 4, 5, 6, 7, 8, or 9 weeks).

An induced Rubus plant (a plant treated with the regimen of (a) and (b)) may be grown under the dormancy conditions of (c) for about 250 hours (about 10 days) to about 40 days (e.g., about 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 550, 555, 560, 565, 570, 575, 580, 585, 590, 595, 600, 605, 610, 615, 620, 625, 630, 635, 640, 645, 650, 655, 650, 655, 660, 665, 670, 675, 680, 685, 690, 695, 700, 705, 710, 715, 720, 725, 730, 735, 740, 745, 750, 755, 750, 755, 760, 765, 770, 775, 780, 785, 790, 800, 810, 805, 810, 815, 820, 825, 630, 835, 840, 845, 850, 855, 850, 855, 860, 865, 870, 875, 880, 885, 890, 895, 900, 905, 910, 915, 920, 925, 930, 935, 940, 945, 950, 955, or 960 hours, or any range or value therein, e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 days or any range or value therein.

In some embodiments, one or more of the conditions of (a) through (c), and when present, (d), may be carried out in a controlled growth environment, optionally wherein the conditions of (a), (b), and (c), and when present (d), may all be carried out in a controlled growth environment.

In some embodiments, the induced Rubus plant of (b) is maintained under dormancy conditions of (c) for a period of time to accumulate chill hours (chill units) sufficient for dormancy and fruit set. As understood in the art, the chill hours (chill units) sufficient for dormancy and fruit set are variety or cultivar dependent, optionally wherein the chill hours sufficient for dormancy and fruit set are about 250 consecutive hours to about 2000 consecutive hours (e.g., about 250, 260, 270, 280, 290, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900 or 2000 consecutive hours, or any range or value therein, e.g., about 10 days to about 85 days, or any range or value therein, e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 consecutive days or any range or value therein, e.g., about 10, 11, 12, 13, 14, 15, 16, 17, 18, 19, 20, 21, 22, 23, 24, 25, 26, 27, 28, 29, or 30 days to about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 consecutive days).

In some embodiments, the Rubus plant is a blackberry variety or cultivar, and the chill hours can be about 300 consecutive hours to about 900 consecutive hours (e.g., about 300, 320, 340, 360, 380, 400, 420, 440, 460, 480 hours to about 500, 550, 600, 650, 700, 750, 800, 850, 900 consecutive hours). In some embodiments, the Rubus plant is a red raspberry variety or cultivar, or a black raspberry variety or cultivar, and the chill hours can be about 250 hours to about 2000 hours (e.g., about 250, 260, 270, 280, 290, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900 or 2000 consecutive hours, or any range or value therein, e.g., about 10 days to about 85 days, or any range or value therein, e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 consecutive days or any range or value therein, e.g., about 10, 11, 12, 13, 14, 15, 16, 17, 18, 19, 20, 21, 22, 23, 24, 25, 26, 27, 28, 29, or 30 consecutive days to about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 consecutive days), optionally the chill hours can be about 600 hours to about 1600 hours (e.g., 600, 650, 700, 750, 800, 850, 900, 950, 1000, 1100, 1200, 1300, 1400, 1500, or 1600 consecutive hours, or any range or value therein, e.g., about 25 consecutive days to about 67 consecutive days, or any range or value therein, e.g., about 25, 26, 27, 28, 29, 30, 31, 32, 33, 34, 35, 36, 37, 38, or 39 days to about 40, 41, 42, 43, 44, 45, 46, 47, 48, 49, 50, 51, 52, 53, 54, 55, 56, 57, 58, 59, 60, 61, 62, 63, 64, 65, 66, or 67 consecutive days).

In some embodiments, the Rubus plant can be any Rubus species, cultivar, or variety. In some embodiments, the Rubus plant can be a floricane fruiting Rubus variety or cultivar (e.g., a plant that naturally flowers and fruits on old wood, e.g., wood produced in the growing season prior to flowering and fruiting) or can be a primocane fruiting Rubus variety or cultivar (e.g., a plant that naturally flowers and fruits on new wood, e.g., wood produced in the same growing season as flowering and fruiting occurs). In some embodiments, the Rubus plant can be a blackberry plant, a black raspberry plant, or a red raspberry plant. In some embodiments, the Rubus plant can be a raspberry (red raspberry) plant including, but not limited to, Rubus crataegifolius (Asian raspberry), Rubus gunnianus (Tasmanian alpine raspberry), Rubus idaeus (red raspberry or European red raspberry), Rubus parvifolius (Australian native raspberry), Rubus phoenicolasius (wine raspberry or wineberry), Rubus rosifolius (Mauritius raspberry), Rubus strigosus (American red raspberry) (syn. R. idaeus var. strigosus), or Rubus ellipticus (yellow Himalayan raspberry). In some embodiments, the Rubus plant can be a black raspberry plant including, but not limited to, Rubus leucodermas (whitebark raspberry or western black raspberry) or Rubus occidentalis (black raspberry). In some embodiments, the Rubus plant can be a blackberry plant including, but not limited to, Rubus argutus, Rubus allegheniensis (Allegheny blackberry), Rubus armeniacus (Himalayan blackberry), Rubus canadensis (Canadian blackberry), Rubus fruticosus agg. (European blackberry), Rubus laciniatus (cutleaf evergreen blackberry), Rubus pensilvanicus (Pennsylvania blackberry), Rubus trifidus (Japanese blackberry), Rubus ursinus (trailing blackberry), or a Rubus hybrid (e.g., progeny resulting from hybridization of two or more of, for example, Rubus allegheniensis, Rubus armeniacus, Rubus canadensis, Rubus fruticosus agg., Rubus laciniatus, Rubus pensilvanicus, Rubus trifidus, and Rubus ursinus). In some embodiments, the Rubus plant can be, but is not limited to, a Rubus chamaemorus (cloudberry) plant, or a Rubus spectabilis (salmonberry) plant.

In some embodiments, when contacting a Rubus plant with the conditions of the invention as described herein, flowering may be achieved in about 120 days to about 14 months (as measured from the time of the emergence of the first true leaves) (e.g., in about 4, 5, 6, 7, 8, 9, 10, 11, 12, 13, or 14 months, or any range or value therein, e.g., about 120, 130, 140, 150, 160, 170, 180, 190, 200, 210, 220, 230, 240, 250, 260, 270, 280, 290, 300, 310, 320, 330, 340, 350, 360, 370, 380, 390, 400, 405, 410, 415, 420, or 425 days, or any range or value therein, or e.g., about 120, 120, 130, 140, 150, 160, 170, 180, 190, 200, 210, 220, 230, 240, 250, 260, 270, 280, 290, or 300 days to about 10, 11, 12, 13, or 14 months). In some embodiments, when applying these methods to a Rubus plant, flowering is achieved/occurs in about 120 days to about 12 months (as measured from the time of the emergence of the first true leaves) (e.g., in about 4, 5, 6, 7, 8, 9, 10, 11, or 12 months, or any range or value therein, e.g., about 120, 130, 140, 150, 160, 170, 180, 190, 200, 210, 220, 230, 240, 250, 260, 270, 280, 290, 300, 310, 320, 330, 340, 350, 360, or 365 days, or any range or value therein, or e.g., about 120, 120, 130, 140, 150, 160, 170, or 180 days to about to about 6, 7, 8, 9, 10, 11, or 12 months). In some embodiments, the methods of the invention applied to a Rubus plant result in the Rubus plant flowering in about 4 months to about 8 months (as measured from the time of the emergence of the first true leaves) (e.g., about 4, 5, 6, 7, or 8 months or any range or value therein, e.g., about 120 days to about 215 days, e.g., about 120, 125, 130, 135, 140, 145, 150, 155, 160, 165, 170, 175, 180, 185, 190, 195, 200, 205, 210, 215, 220, 225, 230, 235, 240 or 245 days or any range or value therein). In some embodiments, the methods of the invention applied to a Rubus plant result in flowering in the Rubus plant in about 5 months to about 7 months (as measured from the time of the emergence of the first true leaves) (e.g., about 5, 6, or 7 months or any range or value therein, e.g., about 120 days to about 215 days, e.g., about 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, or 215 days or any range or value therein), optionally wherein flowering occurs in about 180 days to about 210 days (e.g., about 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, or 210 days, or any range or value therein) from the from the time of the emergence of the first true leaves.

The methods of the invention can reduce the time to flowering in Rubus plants to about 6 months to about 14 months (e.g., about 6, 7, 8, 9, 10, 11, 12, 13, or 14 months, or any range or value therein) as compared to about 18 to about 30 months (e.g., about 18, 19, 20, 21, 22, 23, 24, 25, 26, 27, 28, 29, or 30 months, or any range or value therein) for a control Rubus plant (not grown under the conditions of the invention for reducing time to flowering). In some embodiments, the time to flowering can be reduced by about 30% to about 80% (e.g., about 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80%), as compared to a control Rubus plant (not grown under/exposed to/contacted with the conditions of the invention).

The present invention further provides a Rubus plant and/or a primocane flowering Rubus variety or cultivar produced by the methods of the invention and having a reduced time to flowering. In some embodiments, the time to flowering for a Rubus plant produced by the methods of the invention as described herein can be reduced by about 6 months to about 14 months as compared to a control plant (e.g., the same species, variety, or cultivar as the Rubus plant that is contacted with/exposed to the methods of the invention). In some embodiments, the time to flowering for the Rubus plant may be reduced by about 30% to about 80% as compared to a control.

A primocane flowering Rubus variety or cultivar is a cultivar or variety of any Rubus species that flowers and fruits on primocanes (e.g., naturally flowers on new wood, e.g., wood produced in the same growing season as flowering occurs). The present invention further provides methods for reducing the time to flowering for primocane flowering Rubus varieties or cultivars. In some embodiments, a method of reducing time to flowering of a primocane flowering Rubus variety or cultivar is provided, the method comprising: (a) growing the primocane flowering Rubus variety or cultivar under conditions comprising at least 4 hours of continuous darkness in a 24-hour period and a temperature of at least 21° C. to produce a primocane flowering Rubus variety or cultivar having more than 14 nodes; (b) growing the primocane flowering Rubus variety or cultivar having more than 14 nodes of (a) under induction conditions comprising a temperature of at least 9° C. and a day length of at least 8 continuous hours in a 24-hour period to produce an induced primocane flowering Rubus variety or cultivar; and (c) growing the induced primocane flowering Rubus variety or cultivar of (b) under conditions comprising at least 4 hours of continuous darkness in a 24-hour period and a temperature of at least 21° to produce a primocane flowering Rubus variety or cultivar having flowers, thereby reducing the time to flowering of a primocane flowering Rubus variety or cultivar. In some embodiments, the invention further provides a method of producing a primocane flowering Rubus plant having reduced time to flowering, the method comprising: (a) growing the primocane flowering Rubus variety or cultivar under conditions comprising at least 4 hours of continuous darkness in a 24-hour period and a temperature of at least 21° C. to produce a primocane flowering Rubus variety or cultivar having more than 14 nodes; (b) growing the primocane flowering Rubus variety or cultivar having more than 14 nodes of (a) under induction conditions comprising a temperature of at least 9° C. and a day length of at least 8 continuous hours in a 24-hour period to produce an induced primocane flowering Rubus variety or cultivar; and (c) growing the induced primocane flowering Rubus variety or cultivar of (b) under conditions comprising at least 4 hours of continuous darkness in a 24-hour period and a temperature of at least 21° to produce a primocane flowering Rubus variety or cultivar having flowers, thereby producing a primocane flowering Rubus variety or cultivar having reduced time to flowering.

In some embodiments, the more than 14 nodes on the stem(s) of the primocane flowering Rubus variety or cultivar produced in (b) is about 15 to about 30 nodes (e.g., about 15, 16, 17, 18, 19, 20, 21, 22, 23, 24, 25, 26, 27, 28, 29, or 30 nodes, or any range or value therein). In some embodiments, the at least 4 hours of continuous darkness in (a) and/or (d) may be about 4 hours to about 6 hours of continuous darkness (e.g., about 4, 4.25, 4.5, 4.75, 5, 5.25, 5.5, 5.75, or 6 hours, or any range or value therein of continuous darkness). In some embodiments, the temperature of at least 21° C. in (a) and/or (d) may be a temperature of about 21° C. to about 30° C. (e.g., about 21, 22, 23, 24, 25, 26, 27, 28, 29, 30° C., or any range or value therein).

In some embodiments, the temperature of at least 9° C. in the induction conditions of (b) may be about 9° C. to about 14° C. (e.g., about 9, 10, 11, 12, 13, or 14° C., or any range or value therein). In some embodiments, the day length of at least 8 continuous hours in a 24-hour period for in the induction conditions of (b) may be about 8 hours to about 12 hours (e.g., about 8, 8.25, 8.5, 8.75, 9, 9.25, 9.5, 9.75, 10, 10.25, 10.5, 10.75, 11, 11.25, 11.5, 11.75, or 12 hours, or any range or value therein) in a 24-hour period. In some embodiments, the day length of at least 8 continuous hours in a 24-hour period of (b) (e.g., about 8 hours to about 12 hours) includes light that is supplemented with far-red light (e.g., a wavelength of about 700 nm to about 800 nm).

In some embodiments, the primocane flowering Rubus variety or cultivar is grown under the conditions of (a) for about for about 25 days to about 4 months (e.g., 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days to about 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 114, 112, 113, 114, 115, 116, 117, 118, 191, 120, 121, or 122 days, or any range or value therein, e.g., about 25, 26, 27, 28, 29, 30, 31, 32, 33, 34, 35, 36, 37, 38, 39, 40, 41, 42, 43, 44, 45, 46, 47, 48, 49, 50 days to about 100, 101, 102, 103, 104, 105, 106, 107, 108, 109, 110, 114, 112, 113, 114, 115, 116, 117, 118, 191, 120, 121, or 122 days or any range or value therein, optionally, about 30, 31, 32, 33, 34, 35, 36, 37, 38, or 39 days to about 40, 41, 42, 43, 44, 45, 46, 47, 48, 49, 50, 51, 52, 53, 54, 55, 56, 57, 58, 59, or 60 days, or any range or value therein, and/or is grown under the conditions of (c) for about 1 day to about 127 days (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 124, 125, 126 or 127 days, or any value or range therein, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days to about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 124, 125, 126 or 127 days, or any value or range therein). In some embodiments, the primocane flowering Rubus plants may begin to be exposed to the conditions of (a) at about the time that the first set of true leaves emerge.

In some embodiments, the primocane flowering Rubus variety or cultivar growing under the induction conditions of (b) exhibits exponential growth for about 25 to about 60 days (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, e.g., about 3 weeks to about 2 months). In some embodiments, the primocane flowering Rubus variety or cultivar growing under the induction conditions of (b) is moved to or placed under the conditions of (c) when exponential growth shifts to stationary growth (e.g., when the growth of the primocane flowering Rubus variety or cultivar shifts to stationary growth). As used herein, “exponential growth” is a faster rate of growth than “stationary growth,” wherein the rate of growth is measured as an increase in stem length over time and the increase in stem length over time is greater for exponential growth than for stationary growth. Thus, in some embodiments, the primocane flowering Rubus variety or cultivar is transferred to the dormancy conditions of (c) when the increase in stem length over time of the primocane flowering Rubus variety or cultivar growing under the induction conditions of (b) slows and shifts to stationary growth. As used herein, “exponential growth,” and “stationary growth” for a Rubus plant is measured by selecting at least one stem on the plant and determining the number of nodes that develop on that at least one stem over time. In some embodiments, “exponential growth” of the stem of the primocane flowering Rubus variety or cultivar is production of about one node (e.g., vegetative node) (per stem measured) about every one day to every three days (e.g., in about 1 day, about 2 days, up to about 3 days, e.g., a node is produced about every 72 hours or less; e.g., about every 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 hours, or any range or value therein, e.g., about every 24, 25, 26, 27, 28, 29, 30, 31, 32, 33, 34, or 35 hours to about every 40, 41, 42, 41, 42, 43, 44, 45, 46, 47, 48, 49, 50, 51, 52, 53, 54, 55, 56, 57, 58, 59, 60, 61, 62, 63, 64, 65, or 66 hours, or any range or value therein) per stem and “stationary growth” of the stem of the primocane flowering Rubus variety or cultivar is production of about one node (per stem measured) about every three or more days (e.g. about every 3 days, about every 4 days, about every 5 days, about every 6 days or more (or any combination thereof), e.g., a node is produced about every 72 hours or more, e.g., about every 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 124, 125, 126, 127, 128, 129, 130, 131, 132, 133, 134, 135, 136, 137, 138, 139, 140, 141, 142, 143, or 144 hours, or any range or value therein, e.g., about every 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 124, 125, 126, 127, 128, 129, 130, 131, 132, 133, 134, 135, 136, 137, 138, 139, 140, 141, 142, 143, or 144 hours, or any range or value therein). In some embodiments, a primocane flowering Rubus variety or cultivar growing under the conditions of (b) may be shifted to the dormancy conditions of (c) at about 25 days to about 60 days after being exposed to the inductions conditions of (b) (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, about 25 days to about 9 weeks, e.g., about 3 weeks to about 4, 5, 6, 7, 8, or 9 weeks).

In some embodiments, one or more of the conditions of (a) through (c), may be carried out in a controlled growth environment, optionally wherein the conditions of (a), (b), and (c) may all be carried out in a controlled growth environment.

In some embodiments, the primocane flowering Rubus variety or cultivar can be any Rubus cultivar or variety that flowers naturally on new wood, e.g., wood produced in the same growing season as flowering occurs. In some embodiments, the primocane flowering Rubus variety or cultivar can be a blackberry plant, a black raspberry plant, or a red raspberry plant. In some embodiments, the primocane flowering Rubus variety or cultivar can include, but is not limited to, a cultivar or variety from Rubus idaeus or Rubus argutus.

In some embodiments, when contacting a primocane flowering Rubus variety or cultivar with the conditions of the invention as described herein, flowering may be achieved in about 90 days to about 6 months (as measured from the time of the emergence of the first true leaves) (e.g., in about 3 months to about 6 months, e.g., about 3, 3.25, 3.5, 3.75, 4, 4.25, 4.5, 4.75, 5, 5.25, 5.5, 5.75, or 6 months, or any range or value therein, e.g., about 90, 91, 92, 93, 94, 95, 96, 97, 98, 99, 100, 105, 110, 115, 120, 125, 130, 135, 140, 145, 150, 155, 160, 165, 170, 175, 180, or 185 days, or any range or value therein, or e.g., about 90, 95, 100, 105, 110, 115, or 120 days to about 125, 130, 135, 140, 145, 150, 155, 160, 165, 170, 175, 180, or 185 days).

The methods of the invention can reduce the time to flowering in primocane flowering Rubus varieties or cultivars from 8 months to 12 months (e.g., 8, 9, 10, 11, or 12 months) for a control primocane flowering Rubus variety or cultivar (not grown under the conditions of the invention) to about 3 months to about 6 months (as measured from the time of the emergence of the first true leaves) (e.g., about 3, 3.25, 3.5, 3.75, 4, 4.25, 4.5, 4.75, 5, 5.25, 5.5, 5.75, or 6 months, e.g., about 90 days to about 185 days (e.g., about 90, 91, 92, 93, 94, 95, 96, 97, 98, 99, 100, 105, 110, 115, 120, 125, 130, 135, 140, 145, 150, 155, 160, 165, 170, 175, 180, or 185 days, or any range or value therein, or e.g., about 90, 95, 100, 105, 110, 115, or 120 days to about 125, 130, 135, 140, 145, 150, 155, 160, 165, 170, 175, 180, or 185 days). In some embodiments, the time to flowering can be reduced by about 25% to about 75% (e.g., a reduction of the time to flowering by about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75%).

The present invention further provides a primocane flowering Rubus variety or cultivar produced by the methods of the invention and having a reduced time to flowering. In some embodiments, the time to flowering for the primocane flowering Rubus variety or cultivar produced by the methods of the invention as described herein can be reduced by about 3 months to about 6 months as compared to a control plant (e.g., the same species, variety, or cultivar as the Rubus plant that is contacted with the methods of the invention). In some embodiments, the time to flowering for the primocane flowering Rubus variety or cultivar may be reduced by about 25% to about 75% as compared to a control plant.

The invention will now be described with reference to the following examples. It should be appreciated that these examples are not intended to limit the scope of the claims to the invention but rather are intended to be exemplary of certain embodiments. Any variations in the exemplified methods that occur to the skilled artisan are intended to fall within the scope of the invention.

### EXAMPLES

### Example 1

### Growth Environment

In this example, the controlled environment is a 10×10 grow space capable of providing complete blackout when dark and fully dynamic (tunable) output at 450 nm, 660 nm, 735 nm wavelengths and 5700k (cool white fluorescent light) when illuminated. The grow space is capable of controlled chilling as low as 8° C. in the dark and 11° C. during the day and heating up to a maximum of 40° C. in the day.

Plantlets of the Rubus blackberry varieties set forth in Table 1 (three precommercial varieties and two commercial varieties) were generated as plugs, which were then grown in the growth environment under the conditions of an 18-hour day photoperiod at 28° C., as described in Table 2. This treatment is an example of the conditions of (a) of the methods described herein.

TABLE 1
Rubus plant varieties:

| | | | | |
|---|---|---|---|---|
| Experi- mental/ | Experi- mental/ | Experi- mental/ | Commercial variety #1 | Commercial variety #2 |
| pre- commercial | pre- commercial | pre- commercial | | |
| variety #1 | variety #2 | variety #3 | | |

TABLE 2
Daily light regimen, step (a).

| TIME | 450 NM | 660 NM | 735 NM | 5700K |
|---|---|---|---|---|
| 0:00 | 0 | 0 | 0 | 0 |
| 1:00 | 0 | 0 | 0 | 0 |
| 2:00 | 0 | 0 | 0 | 0 |
| 3:00 | 0 | 0 | 0 | 0 |
| 4:00 | 100 | 100 | 0 | 200 |
| 5:00 | 300 | 300 | 0 | 400 |
| 6:00 | 500 | 500 | 0 | 600 |
| 7:00 | 800 | 800 | 0 | 900 |
| 8:00 | 1000 | 1000 | 0 | 1000 |
| 20:00 | 800 | 800 | 0 | 900 |
| 21:00 | 500 | 500 | 0 | 600 |
| 22:00 | 300 | 300 | 0 | 400 |
| 23:00 | 100 | 100 | 0 | 200 |

0 = lights are off and 1000 = maximum intensity that the lights can give off

Plant development was monitored by measuring the number of nodes and the length of the internodes. The plants were maintained under these conditions until about 15-20 nodes were observed on a stem of plant selected for measuring growth.

The plants with 15-20 nodes were then grown under inductive conditions, which were approximately 9° C. to 14° C. and 8-hour day photoperiod, with the light exposure as outlined in Table 3 for 6 weeks. This treatment is an example of the inducing conditions of (b) of the methods described herein.

TABLE 3
Daily light regimen, step (b).

| TIME | 450 NM | 660 NM | 735 NM | 5700K |
|---|---|---|---|---|
| 0 | 0 | 0 | 0 | 0 |
| 1:00 | 0 | 0 | 0 | 0 |
| 2:00 | 0 | 0 | 0 | 0 |
| 3:00 | 0 | 0 | 0 | 0 |
| 4:00 | 0 | 0 | 0 | 0 |
| 5:00 | 0 | 0 | 0 | 0 |
| 6:00 | 0 | 0 | 0 | 0 |
| 7:00 | 0 | 0 | 0 | 0 |
| 8:00 | 1000 | 1000 | 1000 | 1000 |
| 14:00 | 903 | 660 | 735 | 1000 |
| 14:30 | 767 | 1000 | 1000 | 1000 |
| 14:40 | 627 | 1000 | 1000 | 1000 |
| 14:50 | 489 | 1000 | 1000 | 1000 |
| 15:00 | 358 | 1000 | 1000 | 1000 |
| 15:10 | 243 | 1000 | 1000 | 1000 |
| 15:20 | 151 | 1000 | 1000 | 695 |
| 15:30 | 0 | 200 | 1000 | 695 |
| 15:40 | 0 | 200 | 1000 | 390 |
| 15:50 | 0 | 200 | 1000 | 0 |
| 16:00 | 0 | 200 | 1000 | 0 |
| 16:10 | 0 | 0 | 0 | 0 |
| 17:00 | 0 | 0 | 0 | 0 |
| 18:00 | 0 | 0 | 0 | 0 |
| 19:00 | 0 | 0 | 0 | 0 |
| 20:00 | 0 | 0 | 0 | 0 |
| 21:00 | 0 | 0 | 0 | 0 |
| 22:00 | 0 | 0 | 0 | 0 |
| 23:00 | 0 | 0 | 0 | 0 |

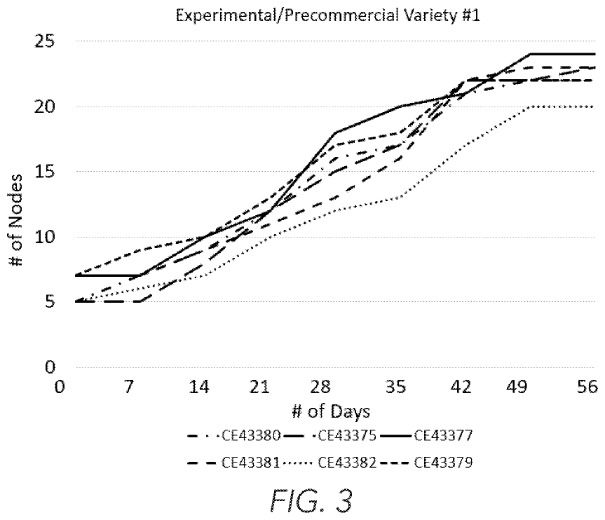

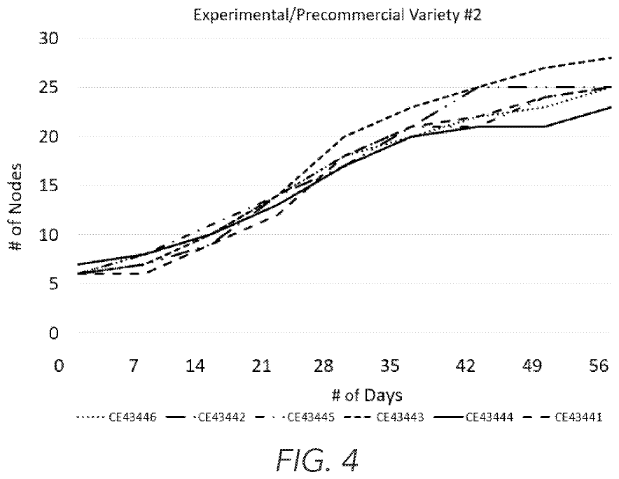

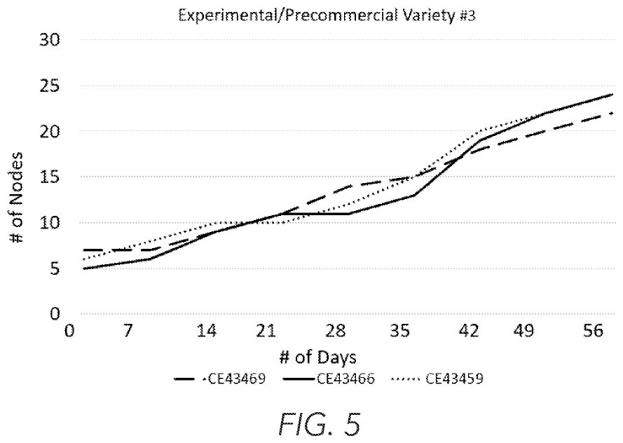

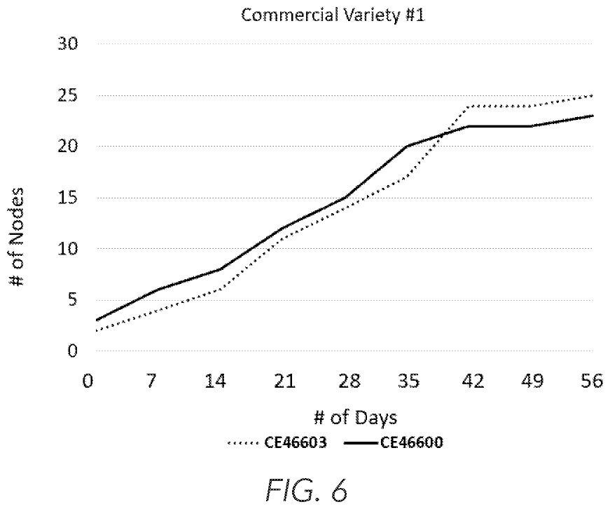

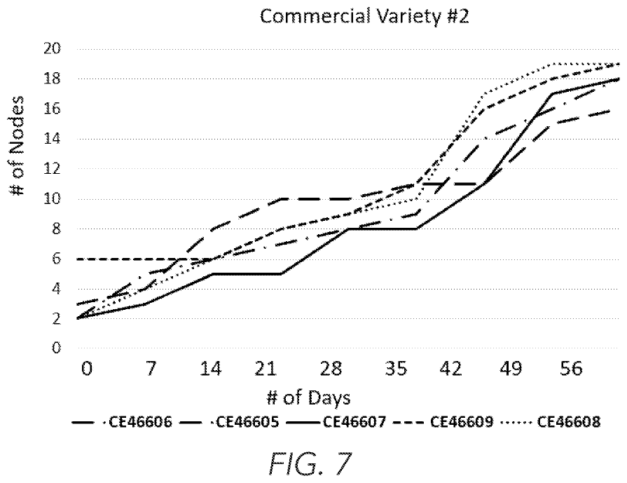

Measurements of nodes produced on a stem selected for measuring the growth of the plant were taken weekly to document the cessation of exponential growth in the inductive environment and the transition to stationary phase growth. Examples of the documentation and transition from exponential growth to stationary growth are provided in FIGS. 3-7.

At about 6 weeks (after cessation of exponential growth), the plants were treated with (exposed to/contacted with) a dormancy period (e.g., the dormancy conditions of (c)) that included about 400 chill hours at 4° C. in the dark (24 hours of continuous darkness).

After completion of dormancy, plants were moved into a constant 16-hour photoperiod with temperatures around 28° C. during the day and 22° C. at night. Flowering time is recorded for the plants.

### Example 2. Rubus occidentalis Rapid Cycling

The goal of this experiment was to apply the methods of rapid cycle as described herein to a black raspberry plant (Rubus occidentalis) and produce flowers. Rubus occidentalis is generally a more northern variety and has longer induction and chill hour requirements for flowering. This experiment demonstrates the methods and conditions as described herein for the induction phase and chill hours can be reliably used to rapid cycle or shorten the time to flowering in black raspberry.

Mature black raspberry plants were generated by germinating seeds and transferring growing seedlings to the greenhouse. After 3 months of growth in the greenhouse (conditions of 16 hour day, 27° C., 8 hour night, 21° C.), the plants were transferred to one of the following environments as shown in Table 4 for induction and chilling.

TABLE 4
Treatment regimens for black raspberry plants
(1) CE101780-8 weeks in induction, 1700 hours cold storage
(2) CE101793-8 weeks in induction, 1700 hours cold storage
(3) CE101790-8 weeks in induction, 1700 hours cold storage
(4) CE101797-12 weeks in induction, 1000 hours cold storage
(5) CE101789-12 weeks in induction, 1000 hours cold storage

The induction conditions for all treatment groups, 1-5 in Table 4, were an 8 hour day at 10° C. and a 16 hour night at 8° C. Cold storage conditions for all treatment groups in Table 4 was no light (zero light) at 5.5° C. Upon completion of the induction and cold storage conditions, plants were returned to the greenhouse (conditions of a 16 hour day, 27° C. day/21° C. night) and monitored for flowering. All treatment groups (1-5) developed flowers within 20 weeks of germinating the seeds.

The foregoing is illustrative of the present invention and is not to be construed as limiting thereof. The invention is defined by the following claims, with equivalents of the claims to be included therein.

1. A method of reducing time to flowering of a Rubus plant, comprising:
(a) growing a Rubus plant under conditions comprising at least 4 hours of continuous darkness in a 24-hour period and a temperature of at least 21° C. to produce a Rubus plant having more than 14 nodes;
(b) growing the Rubus plant having more than 14 nodes of (a) under induction conditions comprising a temperature of at least 9° C. and a day length of at least 8 continuous hours in a 24-hour period to produce an induced Rubus plant; and
(c) placing the induced Rubus plant of (b) under dormancy conditions comprising a 24-hour dark period (i.e., continuous darkness) and a temperature of about 0° C. to about 7° C. to produce a dormant Rubus plant; and/or
(d) optionally, growing the dormant Rubus plant of (c) under conditions comprising at least 4 hours of continuous darkness in a 24-hour period and a temperature of at least 21° C. to produce a Rubus plant that is flowering, thereby reducing the time to flowering of a Rubus plant.

2. The method of claim 1, wherein the at least 4 hours of continuous darkness in (a) and/or (d) is about 4 hours to about 6 hours of continuous darkness, the temperature of at least 21° C. in (a) and/or (d) is about 21° C. to about 30° C., the temperature of at least 9° C. in (b) is about 9° C. to about 14° C., and/or the day length of at least 8 continuous hours in a 24-hour period in (b) is about 8 hours to about 12 hours in a 24-hour period.

3-4. (canceled)

5. The method of claim 1, wherein flowering is achieved in about 120 days to about 14 months.

6. The method of claim 1, wherein flowering is achieved in about 5 months to about 7 months, optionally about 180 days to about 210 days.

7. The method of claim 1, wherein the Rubus plant is grown under the conditions of (a) for about 25 days to about 200 days, the Rubus plant having more than 14 nodes is grown under the induction conditions of (b) for about 30 days to about 60 days and/or the Rubus plant having more than 14 nodes is grown under the dormancy conditions of (c) for about 1 day to about 6 months.

8-9. (canceled)

10. The method of claim 1, wherein when the Rubus plant having more than 14 nodes is grown under the conditions of (d), the Rubus plant is grown under the conditions of (d) for about 1 day to about 6 months.

11. The method of claim 1, wherein the Rubus plant growing under the induction conditions of (b) exhibits exponential stem growth for about 25 to about 60 days.

12. The method of claim 1, wherein the induced Rubus plant is grown under the dormancy conditions of (c) for about 250 hours (about 10 days) to about 40 days.

13-14. (canceled)

15. The method of claim 1, wherein the induced Rubus plant is maintained under dormancy conditions of (c) for a period of time to accumulate chill hours (chill units) sufficient for dormancy and fruit set, the chill hours (chill units) sufficient for dormancy and fruit set are about 250 hours to about 2000 hours, optionally wherein when the Rubus plant is a blackberry variety or cultivar, the chill hours are about 300 hours to about 900 hours and/or the Rubus plant is a red raspberry variety or cultivar or a black raspberry variety or cultivar, the chill hours are about 250 hours to about 1800 hours, optionally about 600 hours to about 1600 hours.

16-24. (canceled)

25. The method of claim 1, wherein the time to flowering is reduced to about 6 months to about 14 months as compared to about 18 to 30 months for a control Rubus plant (not grown under the conditions of the invention) and/or wherein the time to flowering is reduced by about 30% to about 80% as compared to a control Rubus plant.

26. (canceled)

27. A method of reducing time to flowering of a primocane flowering Rubus variety or cultivar, comprising:
(a) growing the primocane flowering Rubus variety or cultivar under conditions comprising at least 4 hours of continuous darkness in a 24-hour period and a temperature of at least 21° C. to produce a primocane flowering Rubus variety or cultivar having more than 14 nodes;
(b) growing the primocane flowering Rubus variety or cultivar having more than 14 nodes of (a) under induction conditions comprising a temperature of at least 9° C. and a day length of at least 8 continuous hours in a 24-hour period to produce an induced primocane flowering Rubus variety or cultivar; and
(c) growing the induced primocane flowering Rubus variety or cultivar of (b) under conditions comprising at least 4 hours of continuous darkness in a 24-hour period and a temperature of at least 21° to produce a primocane flowering Rubus variety or cultivar having flowers, thereby reducing the time to flowering of a primocane flowering Rubus variety or cultivar.

28. The method of claim 27, wherein the at least 4 hours of continuous darkness in (a) and/or in (c) is about 4 hours to about 6 hours of continuous darkness, the temperature of at least 21° C. in (a) and/or in (c) is about 21° C. to about 30° C., the temperature of at least 9° C. in (b) is about 9° C. to about 14° C. and/or the day length of at least 8 continuous hours in a 24-hour period in (b) is about 8 hours to about 12 hours in a 24-hour period.

29-30. (canceled)

31. The method of claim 27, wherein flowering is achieved in about 90 days to about 6 months days.

32. The method of claim 27, wherein the primocane flowering Rubus variety or cultivar is grown under the conditions of (a) for about 25 days to about 4 months.

33. The method of claim 27, wherein the primocane flowering Rubus variety or cultivar having more than 14 nodes is grown under the induction conditions of (b) for about 30 days to about 60 days, and/or the primocane flowering Rubus variety or cultivar having more than 14 nodes is grown under the conditions of (c) for about 1 day to about 127 days.

34. (canceled)

35. The method of claim 27, wherein the primocane flowering Rubus variety or cultivar growing under the induction conditions of (b) exhibits exponential stem growth for about 25 days to about 60 days.

36-38. (canceled)

39. The method of claim 27, wherein the primocane flowering Rubus variety or cultivar is a cultivar or variety of blackberry, black raspberry, or red raspberry, optionally wherein the primocane flowering Rubus variety or cultivar is a cultivar or variety of Rubus idaeus or Rubus argutus.

40. (canceled)

41. The method of claim 27, wherein the time to flowering is reduced to about 3 months to 6 months as compared to about 8 months to 12 months for a control primocane flowering Rubus variety or cultivar (not grown under the conditions of the invention), and/or the time to flowering is reduced by about 25% to about 75% as compared to a control primocane flowering Rubus variety or cultivar.

42. The method of claim 1, wherein the day length of at least 8 continuous hours in a 24-hour period of (b) includes light that is supplemented with far-red light.

43. The method of claim 27, wherein the day length of at least 8 continuous hours in a 24-hour period of (b) includes light that is supplemented with far-red light.

44. A Rubus plant produced by the method of claim 1 and having a reduced time to flowering that is reduced by about 25% to about 80% as compared to a control Rubus plant.

45. A Rubus plant produced by the method of claim 27 and having a reduced time to flowering that is reduced by about 25% to about 80% as compared to a control Rubus plant.

46-90. (canceled)
